SEQUENCE LISTING (27 sequences)

SEQ ID NO: 1 - protein, 10 aa, Hepatitis C virus
YGKAIPLEAI

SEQ ID NO: 2 - protein, 10 aa, Hepatitis C virus
GKAIPLEAIK

SEQ ID NO: 3 - protein, 10 aa, Hepatitis C virus
KAIPLEAIKG

SEQ ID NO: 4 - protein, 10 aa, Hepatitis C virus
AIPLEAIKGG

SEQ ID NO: 5 - protein, 10 aa, Hepatitis C virus
IPLEAIKGGR

SEQ ID NO: 6 - protein, 10 aa, Hepatitis C virus
PLEAIKGGRH

SEQ ID NO: 7 - protein, 10 aa, Hepatitis C virus
LEAIKGGRHL

SEQ ID NO: 8 - protein, 10 aa, Hepatitis C virus
EAIKGGRHLI

SEQ ID NO: 9 - protein, 10 aa, Hepatitis C virus
ELAAKLSGLG

SEQ ID NO: 10 - protein, 10 aa, Hepatitis C virus
LAAKLSGLGI

SEQ ID NO: 11 - protein, 10 aa, Hepatitis C virus
AAKLSGLGIN

SEQ ID NO: 12 - protein, 10 aa, Hepatitis C virus
AKLSGLGINA

SEQ ID NO: 13 - protein, 10 aa, Hepatitis C virus
KLSGLGINAV

SEQ ID NO: 14 - protein, 10 aa, Hepatitis C virus
LSGLGINAVA

SEQ ID NO: 15 - protein, 10 aa, Hepatitis C virus
SGLGINAVAY

SEQ ID NO: 16 - protein, 10 aa, Hepatitis C virus
GLGINAVAYY

SEQ ID NO: 17 - protein, 10 aa, Hepatitis C virus
GLDVSVIPTS

SEQ ID NO: 18 - protein, 10 aa, Hepatitis C virus
LDVSVIPTSG

SEQ ID NO: 19 - protein, 10 aa, Hepatitis C virus
DVSVIPTSGD

SEQ ID NO: 20 - protein, 10 aa, Hepatitis C virus
VSVIPTSGDV

SEQ ID NO: 21 - protein, 10 aa, Hepatitis C virus
SVIPTSGDVV

SEQ ID NO: 22 - protein, 10 aa, Hepatitis C virus
VIPTSGDVVV

SEQ ID NO: 23 - protein, 10 aa, Hepatitis C virus
IPTSGDVVVV

SEQ ID NO: 24 - protein, 10 aa, Hepatitis C virus
PTSGDVVVVA

SEQ ID NO: 25 - protein, 32 aa, Hepatitis C virus
NTNRRPQDVKFPGGGQIVGGVYLLPRRGPRLG

SEQ ID NO: 26 - protein, 24 aa, Hepatitis C virus
AFASRGNHVSPTHYVPESDAAARG

SEQ ID NO: 27 - protein, 24 aa, Epstein Barr Virus
AVDTGSGGGGQPHDTAPRGARKKQ

(19) United States
(12) Patent Application Publication
(10) Pub. No.: US 2004/0235049 A1
(43) Pub. Date: Nov. 25, 2004

(54) Device for presentation of polypeptides able to be used as a chip for miniaturised detection of molecules

(76) Inventors: Oleg Melnyk, Lille (FR); Helene Gras-Masse, Lille (FR); Christophe Olivier, Lille (FR); Jean-Olivier Durand, Lille (FR); Claude Auriault, Lille (FR); Xavier Duburco, Lille (FR); Ahmed Bouzidi, Lille (FR); Jean-Michel Garcia, Lille (FR); Ouafaa El-Mahdi, Lille (FR)

Correspondence Address: BIRCH STEWART KOLASCH & BIRCH, PO BOX 747, FALLS CHURCH, VA, 22040-0747, US

(21) Appl. No.: 10/478,976

(22) PCT Filed: May 27, 2002
(86) PCT No.: PCT/FR02/01771

(30) Foreign Application Priority Data
May 28, 2001 (FR) 0106931

(51) Int. Cl. G01N 33/53; C12M 1/34

(52) U.S. Cl. 435/7.1; 435/287.2

(57) ABSTRACT

The invention concerns a device for presentation of peptides or of proteins, which can be used as a “polypeptide chip” for the miniaturised detection of molecules structurally or functionally complementary to the said polypeptides. This device consists of a flat support onto which the polypeptides are covalently bonded, this bonding between the polypeptides and the support resulting from the formation of a semicarbazone bond. The semicarbazone bond results in particular from the reaction between polypeptides bearing an aldehyde or ketone function and a support functionalised with semicarbazide groups. The invention also concerns the process for preparation of the supports and for attaching polypeptides onto these supports and also the use of the devices thus obtained as polypeptide chips.

### FIELD OF THE INVENTION

The invention concerns a device for presentation of peptides or of proteins, which can be used as a “polypeptide chip” for the miniaturised detection of molecules structurally or functionally complementary to the said polypeptides. This device consists of a flat support onto which the polypeptides are covalently bonded, this bonding between the polypeptides and the support resulting from the formation of semicarbazone bond. The semicarbazone bond results in particular from the reaction between

polypeptides bearing an aldehyde or ketone function

and a support functionalised with semicarbazide groups.

Hereinafter the general term “polypeptide” will be used to designate peptides (comprising at least 2 amino acids, which may be of the L or D series, alpha amino acids, beta amino acids, alpha hydrazino acids, and proteinogenic or nonproteinogenic alpha amino acids), peptido-mimetics (mimics of secondary structure, mimics of beta strand for example) and proteins or fragments of proteins.

The term “polypeptide chip” corresponds to the English terms “peptide arrays, peptide microarrays or peptide chips” common in the literature.

The invention also concerns the process for the preparation of the supports and for attaching polypeptides to these supports and also their utilisation as polypeptide chips.

Such chips are particularly advantageous for the detection, in various liquid biological media, of antibodies or of specific parts thereof, of antigens (especially viral, bacterial or parasitic), of receptors, of sequences responsible for binding to a molecule (enzyme, receptor, antibody), for the study of the specificity of enzymes, for the development of artificial receptors, etc.

Biochips were first created and developed for the detection of nucleic acids. Similar products based on polypeptides are the subject of many studies, but have not yet been optimised. It is therefore above all in the publications and the patents concerning DNA chips that the relevant prior art is found.

Apart from the photolithography technology of Affymetrix and the inkjet technology of Protogene, which make use of the synthesis of oligonucleotides in situ, the production of “microarrays” most commonly uses needle “spotters” and glass slides as supports. The needles sample the probes prepared in the wells of microtitration slides and strike against the surface of the material that serves as the support.

It is now commonly acknowledged that the DNA microarrays created by deposition of probes onto a support suffer from many limitations. Certain are connected with the equipment itself (reproducibility of the volume deposited), but most are the consequence of the organic/inorganic and interface chemistry employed, excessive background noise, sensitivity, immobilisation yield, degradation of the probes due to the various treatments necessary for the immobilisation, stability of the probe-support bond during the various washings and during the microarray recycling stage, quality of the support (uniformity of the treatment, stability with time), non-specific adsorption by the support, and false positives.

In addition to these known problems, connected with the attachment of the nucleotides, the design of polypeptide chips involves specific problems due to the presence, on the polypeptides, of various reactive functions situated on the side-chains of the amino acids which can interfere with the immobilisation methods, which results in binding by multiple bonds or the formation of lateral linkages between polypeptides, which risks decreasing the reactivity of the attached polypeptides towards the biological liquids which are to be tested on these chips.

The methods developed for producing nucleotide chips are not therefore directly transferable to the production of polypeptide chips, the latter requiring the deployment of chemoselective coupling methods.

### DESCRIPTION OF RELATED ART

Various more or less promising coupling methods have already been described.

Thus, the chemistry of thiols has been utilised, but it presents considerable limitations: oxidation of the thiols during their preparation, their storage and their deposition onto the supports (a problem which is all the more serious because during the deposition the area of contact of the film with the air is substantial). For example, this chemistry has been utilised for anchoring peptides of the RGD type onto silica (Porte-Durrieu, M C et al., J. Colloid Interface Science 1990, 134, 368-375), or antibodies onto quartz (Weiping, Q. et al., Supramolecular Science 1998, 5, 701-703, formation of disulphides). The formation of thioethers, and the formation of the Hg-S bond, may also be cited (Rao, S. V. et al. Mikrochim. Acta 1998, 128, 127-143).

Many laboratories have immobilised proteins via the formation of a hydrazone bond between an aldehyde and a hydrazide. However, these methods also present many limitations. The hydrazone linkage is not very stable and hydrolyses rapidly (King, T P et al., Biochemistry 1986, 25, 5774-5579). For this reason, many authors reduce this bond with a borohydride to stabilise the linkage (see for example King, T P et al., Biochemistry 1986, 25, 5774-5579, Ruhn, P. F. et al., J. Chromatography A 1994, 669, 9-19). Furthermore, the reaction of a hydrazide with an aldehyde is relatively slow.

Falipou, S. et al. (Bioconjugate Chem 1999, 10, 346-53) describe the silanisation of SiO₂ beads or glass slides with 3-cyanopropyldimethylchlorosilane. The surfaces thus treated make it possible to immobilise antibodies (via the hydroxyls of the glycosylated parts) non-covalently.

In the patent US 4874813, it is stated that the coupling of IgG (after periodate oxidation to generate an aldehyde function) to a hydrazide support is effected with a yield of only 53%. This chemistry is not well suited to biochip technology, where the reactions have to be very rapid in order to compensate for the low concentration of substance to be attached and its mobility during the drying of the deposit. In the same patent, it is stated that the incorporation of a tertiary amine (pKa<8) at the level of the support makes it possible to accelerate the bonding to the support. Typically this is an acceleration due to the non-specific interactions induced by the ammonium. This phenomenon is thoroughly discussed by Robberson et al. (Biochemistry 1972, 11, 533-536) in the context of the immobilisation of RNA on a gel of agarose hydrazide, and is probably due to the modification of the diffusion properties of the biomolecules due to the reduction in size (Adam, G. et al., 1968, in Structural Chemistry and Molecular Biology, Rich, A., Davidson, N. Ed., San Francisco, W H Freeman). However, such non-specific adsorption is precisely what must as far as possible be avoided in the context of biochips.

The immobilisation of periodate-oxidised IgG on a silica hydrazide is described by Ruhn, A F et al. (J. Chromatography A 1994, 669, 9-19). The reaction takes 1 day at 4° C.; this support decomposes at higher temperature and gives rise to considerable non-specific adsorption which rises to 20% of the value obtained by covalent bonding. This type of chemistry is not favourable for the production of biochips.

In the patent U.S. Pat. 4,890,1726, a (silica) hydrazide support for the immobilisation of toxins of a non-peptide nature by a non-hydrazone bond is described. The support (only silica gels, no flat surfaces) is prepared by reaction with an epoxide silane then opening of the epoxide with a dihydrazide. Now, the reaction of dinucleophiles on a support is known to give rise to many bridging reactions. This point is well documented in the article by Ruhn, P F et al., J. Chromatography A 1994, 669, 9-19. Thus, the surface function density is much decreased. The bridging reactions are also the source of reproducibility problems.

Many studies make use of the in situ synthesis of polypeptides on the support:

Ashfield, C. et al. “Synthesis of peptides in picoliter virtual flasks” (16^{th} American Peptide Symposium Minneapolis, Minn., Jun. 26 - Jul. 1, 1999 Poster 823) describe the in situ synthesis of peptides on semiconductor. Electrodes are covered with a porous polymer, within which the peptide synthesis is carried out. A linker containing a —NH-Fmoc function is bound to the polymer. The Fmoc group is removed by generating a base in situ through the application of a redox potential. The amine function thus liberated is reacted with activated amino acids by normal means.

Pellois, J. P. et al. (J. Comb. Chem. 2000, 2, 355-360) describe the in situ synthesis on a glass slide.

The basic chemistry is of the Boc/benzyl type. The glass slide is amine silanised, then the first amino acid is coupled by normal means. The deprotection of the tBoc group is effected by irradiation (400 nm) in the presence of triarylsulphonium hexafluoroantimonate or diaryl iodium hexafluoroantimonate. The coupling of the next amino acid is effected by normal means.

In the patent WO 0053625, a mode of in situ biochip synthesis is described.

A porous polymer is deposited on a series of electrodes. In an initial period, an Fmoc amino acid-OH is coupled to the porous polymer. The Fmoc group is removed by the application of a potential to a given electrode in the presence of azobenzene. The biochip is then immersed in a solution containing the next activated amino acid. In situ synthesis suffers from a large number of limitations:

it necessitates the use of photolabile protective groups or of reagents activated by the application of an electrical potential;

this technology is currently of low efficacy (limitation of the size of the peptides, high cost, low flexibility—see comments by Pellois, J. P., J. Comb. Chem. 2000, 2, 355-360),

the characterisation of the peptides bound to the support is difficult.

In an entirely different field, the utilisation of beads of polystyrene resin functionalised with semicarbazide functions has been described for carrying out the synthesis, step by step, of aldehyde peptides:

Siev, D V et al. (Org. Lett. 2000, 2, 19-22) describe a hydrazino-carbonyl-aminomethylated polystyrene resin (beads) for the synthesis of aldehyde peptides and peptidomimetics.

Patterson, J A et al. (Tetrahedron Lett. 1999, 40, 6121-6124) describe, starting from a resin of the aminated polystyrene type, the formation of the isocyanate with triphosgene and the reaction of the isocyanate with Fmoc—NH—NH₂ (prepared after Fhang, Z. E. et al., Anal. Biochem. 1991, 195, 160-170). The support serves for the immobilisation of aminated aldehydes, then for the solid phase synthesis of aldehyde peptides protected in semicarbazide form. The semicarbazone is exchanged in solution with pyruvic acid to liberate the aldehyde peptide.

Murphy, A M et al. (J. Am. Chem. Soc. 1992, 114, 3156-3157) described the reaction of an aminated aldehyde in homogenous phase with a semicarbazide functionalised with a carboxylic acid function. After the formation of the semicarbazone, the synthon is anchored to a solid support (beads) and serves for the solid phase synthesis of aldehyde peptides.

A significant advance in the development of peptide biochips was presented by Falsey et al. (16^{th} American Peptide Symposium—Minneapolis 1999—Poster No. 822). These authors bound peptides functionalised with a hydroxylamine H₂N-O- or a beta-amino thiol (N-terminal cysteine) to glass slides functionalised with an alpha-oxo aldehyde function. The glass slide was amine silanised, then the amine functions were derivatised with an Fmoc-Ser-OH. After deprotection of the Fmoc group with piperidine, the slides were treated with sodium periodate (oxidation of the beta amino alcohol to alpha-oxo aldehyde). The major disadvantage of this method is the non-specific adsorption of polypeptides at the surface, leading to substantial background noise.

The Applicants have sought to resolve the principal limitations of the devices previously developed, and in particular:

the background noise problems due to the non-specific adsorption of the samples to be tested;

the excessively weak or irregular functionalisation of the surface of the support;

the low efficiency anchoring of the probes (polypeptides).

### SUMMARY OF THE INVENTION

Thus, the devices according to the invention comprise flat supports, of solid material, organic or inorganic which may for example be of glass, of silicon or derivatives thereof, of natural or synthetic polymer, presenting a flat surface functionalised with a group such as a semicarbazide group for immobilising selected polypeptides in a controlled manner utilising the formation of a semicarbazone linkage. This is obtained by the utilisation of polypeptides previously functionalised with an aldehyde or ketone group, preferably alpha-oxo aldehyde or alpha-oxo ketone.

In the case of synthetic polypeptides, the aldehyde or ketone group is introduced in the course of synthesis either at the N-terminal end, or at the C-terminal end, or on a side-chain (of Lys or Cys).

In the case of natural glycosylated polypeptides, aldehyde groups are generated by oxidation of the polysaccharide resides.

In the case of natural (whether or not glycosylated) polypeptides (fragments of proteins), a ketone or aldehyde function can be generated by transamination of the N-terminal residue or an aldehyde or ketone function by the use of a bifunctional reagent. The aldehyde or ketone function can be located on a spacer arm.

The deposition of the functionalised polypeptides onto the semicarbazide support is attended by the spontaneous bonding of the peptide to the support, under selected pH, temperature and humidity conditions.

The semicarbazide slides can for example be printed with a needle “spotter”. After the deposition of the polypeptides onto the slide, this can be immersed, when this is necessary, in a solution containing a polyethylene glycol derivatised with an α-oxo aldehyde or ketone function. Thus, all the reactive sites present between the spots are covalently bound to a PEG, via the same semicarbazone linkage used for immobilising the polypeptides, which further reduces the non-specific adsorption of the test samples.

Thus the immobilisation strategy

is simple at the experimental level and highly reproducible;

applies for polyfunctional peptides, including polypeptides containing cysteines;

utilises polypeptides modified with a stable function which is easy to introduce;

utilises surfaces functionalised with a stable, non-hydrolysable function;

makes use of very reactive functions, compensating for the low concentration of the polypeptides at the deposition level;

makes it possible to obtain a high surface attachment density which will ensure a very high signal to noise ratio;

respects the structure of the polypeptide (specific bonding to the support by a single anchoring point);

allows quality control at all stages.

The chemistry of hydrazone ligation has been greatly developed in the field of protein engineering for the convergent synthesis of macromolecules based on the controlled linking of totally deprotected and purified fragments. The two functional groups, present on each of the fragments, mutually recognise one another with high selectivity under very mild conditions (see for example J. P. Tam et al., Biomed. Peptide Protein & Nucleic Acids 1995, 1, 123-132). The semicarbazone linkages are much more stable than hydrazones and for this reason were chosen for the attachment of the polypeptides according to the present invention.

Further, it has surprisingly been found that the binding of the semicarbazides onto the support took place densely, homogeneously and reproducibly. This density of functionalisation has been found to be one of the keys to the success of the biochips according to the invention: indeed it increases the sensitivity of the test which is essential for working with microquantities and thus makes it possible to measure responses (probe-target, antigen-antibody) without having recourse to amplification strategies. It also ensures extremely low background noise and hence a very high signal to noise ratio which increases the sensitivity of the test and will be particularly advantageous in the case of tests involving biological liquids very rich in different proteins.

The quality of the functionalisation of the support according to the invention (density and homogeneity) can be checked by its capacity to immobilise a synthetic, fluorescent peptide probe, derivatised with an (α-oxo aldehyde function.

The invention also concerns the process for preparation of devices for presentation of polypeptides which comprises the following stages:

1. introduction of an aldehyde or ketone function, by synthesis or by modification of a natural function, at one of the ends, N or C, or on a side-chain, of a synthetic or natural polypeptide;

2. functionalisation of a solid support with semicarbazide groups;

3. deposition in the form of spots, of samples of polypeptides obtained by stage 1 onto the support functionalised in stage 2, under pH and humidity conditions ensuring the reaction between the aldehyde or ketone function and the semicarbazide function to create the semicarbazone linkage.

Stage 1 can be effected in the course of the synthesis of a polypeptide using an automatic synthesiser. It can include the introduction of a spacer arm between the last amino acid of the polypeptide sequence and the aldehyde or ketone function.

Stage 1 can be effected by oxidation of a polysaccharide of a natural glycoprotein or of a fragment thereof or by transamination of an N-terminal amino acid of a natural non-glycolysated protein or of a fragment thereof, or by the action of a bifunctional reagent.

Stage 2 comprises

a reaction of silanisation of the support, introducing an amine function;

the transformation of the amine function into an isocyanate function;

the reaction of the isocyanate function with a hydrazine derivative to form the semicarbazide group.

Alternatively, stage 2 can be effected in a single reaction of a silane bearing a semicarbazide group, which is preferably protected with Fmoc.

Stage 3 preferably comprises

the preparation of 10⁻³ to 10⁻⁴ M solutions of the polypeptides from stage 1 in an 0.1M acetate buffer at pH 5.5,

their distribution in a receptacle appropriate for their sampling, of the microtitration well slide type,

their sampling using a “spotter”,

and their deposition onto the semicarbazide support;

the incubation of the slides for one night at 37° C. under a moist atmosphere

and their washing and the-saturation of the non-specific reactive sites.

The invention also concerns the utilisation of the devices for presentation of polypeptides as “polypeptide chips” as a diagnostic tool. This utilisation comprises the detection of responses of the antigen-antibody type by the utilisation of labelled, fluorescent, radioactive or chemically labelled reagents, as in non-miniaturised diagnostic tests.

The devices according to the invention can also be utilised as polypeptide chips for the screening of molecules and for the analysis of the relationships between molecules, of the ligand-receptor type.

The examples which follow illustrate the invention without however limiting its scope.

### BRIEF DESCRIPTION OF THE DRAWINGS

The following diagrams are appended thereto:

FIG. 1: comparative test of serums on the peptide HCVpc21-2 by ELISA (measurement of the o.d.) and on semicarbazide slides (measurement of the fluorescence/20000).

FIG. 2: study of the correlation between the o.d. and fluorescence measurements presented in FIG. 1.

FIG. 3: comparative test of sera on 3 HCVpc21 peptides “small linker”, “large linker” and N-terminal (HCVpc21 3, 2 and 1); measurement of fluorescence.

FIG. 4: comparison of the responses of 30 positive sera with the peptides HCVpc21, NS4 and a pc21/NS4 mixture (1 to 10); measurement of fluorescence.

### BRIEF DESCRIPTION OF THE PREFERRED EMBODIMENTS

### EXAMPLE 1

### Functionalisation of Glass Slides

The covalent binding of semicarbazide groups onto the glass slides was effected in accordance with 2 strategies.

First Strategy

Stage A: washing, stripping and silanisation

Commercial microscope slides (Esco), prewashed, with ground edges and frosted rims are immersed in a “piranha” solution (50/50 hydrogen peroxide/sulphuric acid) for one night. Preliminary three minute rinses are carried out with deionised water (3 times) then with methanol (once), before immersing the slides in a bath of 3% aminopropyl-trimethoxysilane in 95% methanol for 30 minutes with ultrasonication. The slides are rinsed successively by 3 minute immersions in methanol (once), deionised water (twice) and finally methanol (once). The slides are then drained for a few minutes, dried for 15 minutes in an oven at 110° C. then stored under vacuum in a desiccator.

Stage B: Formation of an Isocyanate

The previously silanised slides are immersed for 2 hours in a solution of 1,2-dichloroethane containing triphosgene (100 mmoles/l) and DIEA (800 mmoles/l).

Stage C: Ffunctionalisation with a Semicarbazide

These slides are then rapidly drained before being directly immersed in the 22 mmole/l solution in DMF containing Fmoc—NH—NH₂ (prepared after Zhang et al., Anal. Biochem. 1991, 195, 160-170) and ultrasonicated for 2 hours.

The slides are then rinsed successively with two 3 minute immersions in DMF.

Stage D: Deprotection

The slides obtained as above are immersed in a solution of DMF containing piperidine (0.2% by volume) and diazabicyclo-undecene (2% by volume) for 30 minutes. The slides are then rinsed successively by immersion for 3 minutes in DMF (once), with deionised water (twice) and finally with methanol (once) before being dried and stored under vacuum in a desiccator.

Second Strategy

The silanisation and semicarbazide functionalisation stages are coupled in a single reaction, by prior preparation of the reagent.

Stage A: preparation of the reagent Fmoc-NH—NH—CO—NH—(CH₂)₃—Si(OEt)₃.

The preparation of the reagent is carried out in accordance with the following diagram:

1) Preparation of the Reagent

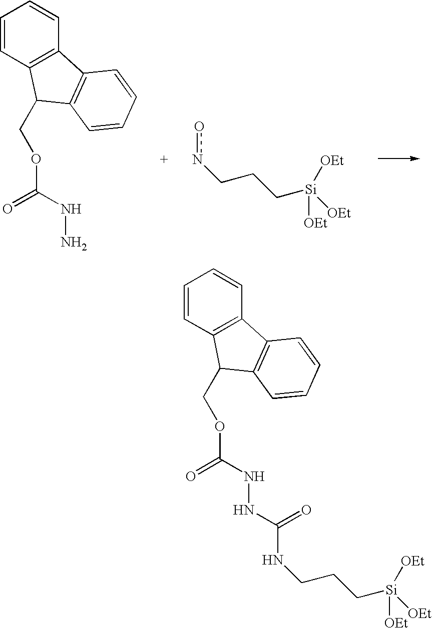

515 mg of Fmoc-NHNH₂ (2.03 mmoles) are suspended in 15 ml of absolute ethanol. The mixture is heated to reflux (75-80° C.). 570 ml of isocyanopropyltriethoxysilane (2.28 mmoles, 1.2 eq.) are then added all at once. After disappearance of the suspension, (15-20 minutes), the ethanol is evaporated. The white solid is dissolved in a minimum of dry dichloromethane, then precipitated with dry pentane. After filtration under argon, 841 mg (83%) of a pure solid are recovered.

Stage B: Preparation of the Slides

Prewashed microscope slides (Esco), with ground edges and frosted rims are immersed for one night with stirring in a freshly prepared solution of piranha (H₂SO₄/H₂O₂). The slides are then successively rinsed with stirring in the following baths: deionised water (3×3 minutes) and absolute ethanol (1×3 minutes), then dried at the vane pump.

They are then immersed for 2 hours in a 1 mg/ml solution of silanisation reagent (Fmoc-NH —NH—CO—NH—(CH₂)₃-Si(OEt)₃) in a 10% mixture of THF in toluene at 47° C. and with ultra-sonication. The slides are then rinsed with stirring in toluene (2×3 minutes) before being drained, then dried for 15 minutes in an oven at 120° C. and stored under vacuum in a desiccator.

Stage C: Deprotection

The slides obtained above are immersed for 3 minutes in a solution of DMF before being placed with stirring in a bath containing piperidine (0.2% by volume) and diazabicyclo-undecene (2% by volume) in DMF for 30 minutes. The slides are then rinsed successively by immersion for 3 minutes in DMF (1×3 minutes), with deionised water (2×3 minutes) and finally with methanol (1×3 minutes) before being dried and stored under vacuum in a desiccator.

### EXAMPLE 2

### Checking of Quality of Semicarbazide Slides

It is important to check that the functionalisation of the slides is homogeneous and reproducible in order to guarantee homogeneous attachment of the polypeptides.

The quality of the slides is thus checked using the same reaction as that which will be used to attach the polypeptides. A small synthetic peptide functionalised with an α-oxo aldehyde group and labelled with rhodamine (fluorescent marker) is used, and, as negative control, the same peptide in which the α-oxo aldehyde is replaced by an amide group. The rhodamine-conjugated peptide functionalised with an α-oxo aldehyde group of sequence (5)-6-carboxytetramethylrhodamine-Lys-Arg-NH—(CH₂)₃—NH—CO—CHO was synthesised from the linker PT (2,3-O-isopropylidene D-tartrate), described by J. S. Fruchart et al., H. Gras-Masse, O. Melnyk (A new linker for the synthesis of C-termninal peptid α-oxo aldehydes, Tet. Lett., 40, 6225-6228, 1999), and in the patent applicatio PCT/FR00/01035.

As the control peptide, (5)-6-carboxytetramethyl-rhodamine-Lys-Arg-NH₂ is synthesised.

Development:

The slides fuinctionalised with a semicarbazide group are immersed for I hour at 37° C. in a bath of rhodamine-conjugated peptide (functionalised on the C-termninal side with an α-oxo aldehyde or non-functionalised control) at a concentration of 0.1 mM in a 100 mM acetate buffer and at pH 5.5. The slides are then rinsed by passing through a bath of deionised water. They are then transfered into a 5% solution of K₂HPO₄ in water for 2 hours and ultrasonicated. The slides are then rinsed by immersion with stirring in deionised water (3 minutes twice) then are immersed for 30 minutes w with ultrasonication in a solution of Tris acetate (100 mM) at pH 5.5 in the presence of Tween 20 (0.1%). The slides are then rinsed successively by immersion in deionised water (3 minutes twice) and finally in methanol (3 minutes once). The slides are then dried under vacuum in a desiccator.

The slides are then passed through the MWG scanner (L30 PMT45). The fluorescence of the whole slide is quantified, using a grid of 32 lines and 10 columns of spots on the software Scanlyse. The fluorescence values are shown in the following table:

| | Type of slide | | | | | |
|---|---|---|---|---|---|---|
| | Functionalised slides | | | | | |
| | First | | Second | | | |
| | modification | | modification | | Non-functionalised | |
| | α-oxo | | α-oxo | | slides | |
| Type | alde- | | alde- | | α-oxo | |
| of probe | hyde | amide | hyde | amide | aldehyde | amide |
| Mean | 36260 | 3450 | 28882 | 1732 | 7789 | 5270 |
| fluorescence | | | | | | |
| Standard | 3939 | 486 | 3427 | 91 | 671 | 934 |
| deviation | | | | | | |

### EXAMPLE 3

### Synthesis of Test Peptides

The following peptides were synthesised (on an automatic synthesiser of the Pioneer PerSeptive Biosystem type).

1—Peptides of the Hepatitis C Virus

HCVpc21-1 peptide functionalised with an α-oxo aldehyde group on the N-terminal side:

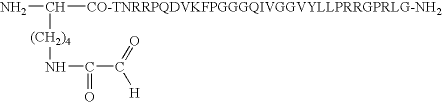

This peptide is solid phase synthesised from the C-terminal end towards the N-terminal end on the basis of an Fmoc strategy, on an Fmoc-PAL-PEG-PS resin (Perseptive Biosystems).

HCVpc21-2 peptide functionalised with a 4,7,10-trioxa-1,13-diamino-tridecanyl-α-oxo aldehyde group on the C-terminal side, “large linker”:

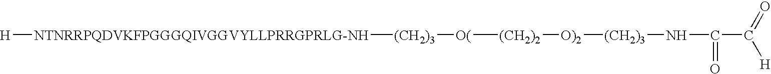

This peptide is solid phase synthesised from the C-terminal end towards the N-terminal end on the basis of an Fmoc strategy, on a “methyl-2,3-O-isopropylidene-D-tartryl-Val-PEGA resin, prepared as described in “Peptides for the new millenium”, Proceeding of the 16^{th} American peptide symposium, (kluwer academic publishers, Dordrecht, 2000, p.104-106). 0.1 mole of resin are conditioned in a solid phase reactor by washing with DCM (2 minutes twice) then DMF (2 minutes twice). A mixture of 1.688 ml of 4,7,10-trioxa-1,13-tridecane-diamine (7.7 mmoles) and DFM (812 μl) is then added onto the almost dry resin. After stirring for 45 minutes, the resin is washed with two successive DMF washings. The first amino acid of the sequence is coupled in the reactor and 10 eq. of the following reagents are added to the almost dry resin: Fmoc-Gly-OH, HBTU, HOBt and 30 eq. of DIEA in DMF (2.5 ml). The solution is stirred for 45 minutes before being washed with DMF (4×2 min) then DCM (4×2 min). The following amino acids are then grafted on by transferring the residue obtained to an automatic peptide synthesiser of the Pioneer PerSeptive Biosystem type.

HCVpc21-3 peptide functionalised with a 1,3-diaminopropyl-α-oxo aldehyde group on the C-terminal side, “small linker”:

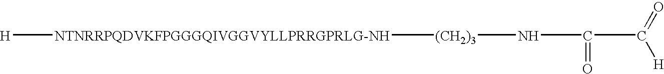

This peptide is solid phase synthesised from the C-terminal end towards the N-terminal end on the basis of an Fmoc strategy, on a methyl-2,3-O-isopropylidene-D-tartryl-Val-PEGA resin. 0.1 mmole of resin are conditioned in a solid phase reactor by washing with DCM (2×2 minutes) then DMF (2×2 minutes). A mixture of 649 μl of 1,3-diaminopropane (7.7 mmoles) and DMF (351 μl) is then added onto the almost dry resin. After stirring for 20 minutes, the resin is washed with two successive DMF washings. 10 eq. of the following reagents are then added to the almost dry resin: Fmoc-Gly-OH, HBTU, HOBt and 30 eq. of DIEA in DMF. The solution is stirred for 45 minutes before being washed with DMF (4×2 min) then DCM (4×2 min). The other amino acids are then grafted on successively.

Derivative of the peptide NS4 functionalised with a 4,7,10-trioxa-1,13-diamino- tridecanyl-α-oxo aldehyde group on the C-terminal side:

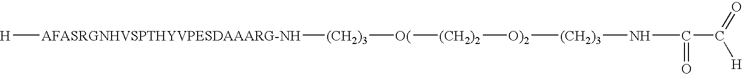

This peptide is solid phase synthesised from the C-terminal end towards the N-terminal end on the basis of an Fmoc strategy, on a methyl-2,3-O-isopropylidene-D-tartryl-Val-PEGA resin. The synthesis is carried out according to the same protocol as that for the peptide HCVpc21-2.

2. Peptide of the EBV Virus

Derivative of the peptide VCA p18 (EBV) functionalised with a 4,7,10-trioxa-1,13-diamino -tridecanyl-α-oxo aldehyde group on the C-terminal side:

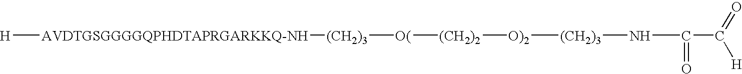

This peptide is solid phase synthesised from the C-terminal end towards the N-terminal end on the basis of an Fmoc strategy, on the resin methyl-2,3-O-isopropylidene-D-tartryl-Val-PEGA following the same protocol as for the peptide HCVpc21-2.

### EXAMPLE 4

### Protocol for Utilisation of the Semicarbazide Slides

1. Ligation of Peptides

The functionalised peptides are firstly solubilised (at 10⁻³ M or 10⁻⁴ M) in 0.1 M acetate buffer, pH 5.5. They are then distributed into the wells of a 384-well ELISA plate (Microtest TM, Becton Dickinson, N.J. USA). By means of a manual “Spotter” with 32 needles (Microarray Printer XMM 47832-Xenopore, Hawthorne, US), the peptides are sampled from the ELISA plate and deposited onto a semicarbazide glass slide. The slides are then placed to incubate for 1 night at 37° C. under a moist atmosphere.

These slides are then washed, by passage for 60 mins with ultrasonication in a solution of Tris acetate (0.IM tris(hydroxymethyl)-aminomethane—Merck, Darmstadt, Germany). The slides are then washed 4 times for 3 minutes in a solution of PBS (0.01M phosphate buffer with 1.8% added NaCl, pH 7.4) in the presence of 0.05% of Tween 20.

2. Contacting with the Test Sera

The slides are then incubated in the presence of 100 μl of serum from patients diluted to 1/50^{th} in the dilution buffer (PBS+2.5% of powdered semi-skimmed milk+0.5% of Tween 20) under a glass cover slip (24×60 mm). The incubation of the slides is effected for 2 hours at 37° C. under a moist atmosphere. 4 successive washes are then effected for 3 minutes in PBS solution with 0.05% added Tween 20.

3. Detection of Specific Antibodies in the Sera by Measurement of Fluorescence

The reaction of the antibodies of patients with the polypeptides attached to the slide is detected by the binding onto these of fluorescent anti-human IgG antibodies. 100 μl of solution of anti-human IgG-A-M antibodies labelled with rhodamine (TRITC) (Jackson ImmunoResearch Laboratories, Baltimore, US) diluted to 1/100^{th} in the dilution buffer (PBS+2.5% of powdered semi-skimmed milk+0.5% of Tween 20) are then deposited onto each slide. Each slide is then covered with a cover slip (24×60 mm) and left to incubate for 1 hours at 37° C. under a moist atmosphere. A series of 4 successive washes in PBS solution with 0.05% of added Tween 20 is carried out. The slides are then rinsed using a mQ water wash-bottle then 95° ethanol for 1 minute, then dried for 15 minutes in ambient air.

The fluorescence emitted is then detected using a slide scanner (L35/PMT 50, Affymetrix 418 Chip Scanner, MWG).

### EXAMPLE 5

### Utilisation of the Slides as Polypeptide “Chips” for the Detection of Antibodies Present in the Sera

1. Study on the Peptide HCVpc21

Sera from patients whose serology is known (that is to say verified by reference tests) were compared in an ELISA test and with the “biochips” according to the invention:

30 positive sera numbered from P1 to P30

10 negative sera numbered from N1 to N10

The slides according to the invention are utilised with 16 spots of the peptide HCVpc21-2 (described in Example 3).

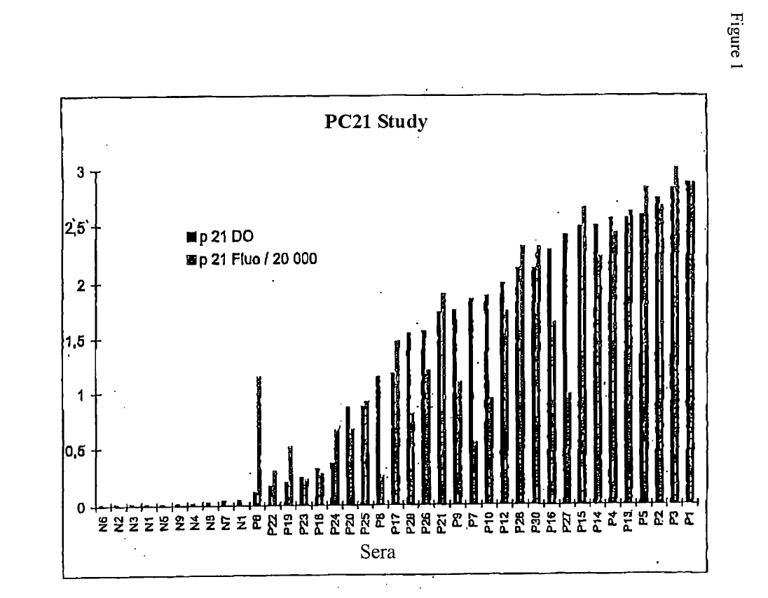

The ELISA tests are performed on COSTAR carbobind plates, with the same peptide HCVpc2l1-2. The results of the ELISA test, expressed in optical density, and the results of the “biochips” test, expressed in fluorescence/20000 (mean of the measurement of the fluorescence of the 16 spots per sample) are shown in FIG. 1.

The biochips test was found to be sensitive and 100% specific for this study on these referenced HCV sera, since it made it possible to detect all the positives and no negative.

2. Comparison Between the Optical Density Obtained with the ELISA Test and the Fluorescence of the Semicarbazide Slides for the Peptide HCVpc21

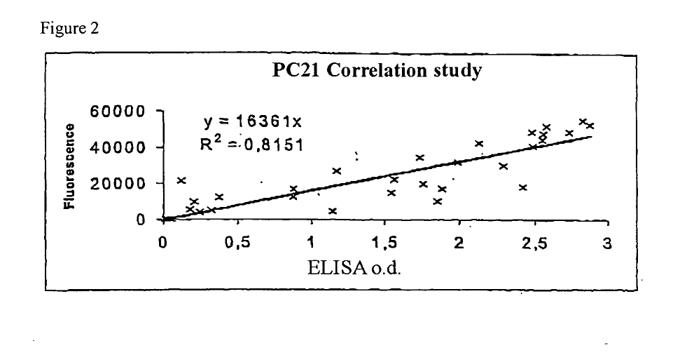

The results of the above study were plotted in FIG. 2 which shows the correlation between fluorescence and optical density.

No fluorescence (fluorescence corresponding to the background noise) is observed for the negative sera, which is not the case for the ELISA where the negatives have a value. Once a fluorescence is observed with the biochip technique, it is known that it is a positive, which is not the case with the ELISA where a low o.d. value can correspond to a negative. With ELISA, this involves the definition of a threshold value which corresponds to the mean of the negative sera+3 times their standard deviation; with the biochips, the threshold value is the mean of the background noise+3 times the standard deviation, which in contrast to the ELISA corresponds to a very low value. Consequently, the differential between negative and positive sera is much greater for the biochips than for the ELISA.

Furthermore, it is also possible to increase the gain in sensitivity by increasing the power of the scanner. Thus a fluorescence value corresponding to the background noise is still retained for the negativ sera, however the fluorescence value of the positive spots is increased; this is especially interesting in the case of weakly positive sera which are then detected without any ambiguity.

3. Reproducibility Study

The following table expresses the mean fluorescence observed on 9 different slides, towards the same serum identified as HCV serum by the ELISA test. On each slide, 120 spots of the peptide HCVpc21-2 “large linker” at the concentration of 10⁻⁴ M were made. The mean fluorescence observed on the 9 slides is 41095.

The standard deviation on the 9 slides is 1891, i.e. a standard deviation of 4.5%.

TABLE 1

| Slide No. | Fluorescence |
|---|---|
| L1 | 42104 |
| L2 | 40282 |
| L3 | 37676 |
| L4 | 40853 |
| L5 | 40337 |
| L6 | 42375 |
| L7 | 39568 |
| L8 | 43918 |
| L9 | 42734 |

4. Comparison Between the Different Types of HCVpc21 Peptides

This study was performed on 5 different slides (5 different sera; 1 serum per slide, HCV sera positive by the ELISA carbobind HCVpc21 test and by the Abbott EIA 3.0 test, currently on the market).

On each slide, 8 spots per HCVpc21 peptide were deposited at the concentration of 10⁻⁴ M.

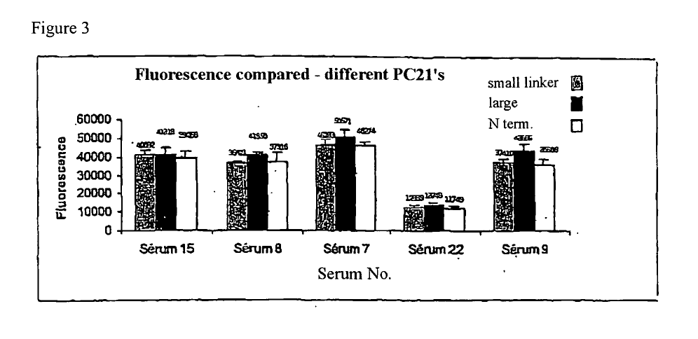

3 different types of peptide (such as described in example 3) were studied, HCVpc21-1, HCVpc21-2 and HCVpc21-3. The results are presented in FIG. 3.

The fluorescence is higher with the HCV peptide “large linker”. The results are comparable for the “small linker” peptide and HCVpc21-1. The mean gain in fluorescence over the 5 slides is 9.9% in favour of the “large linker” relative to the “small linker” and is 11.11% for the “large linker” relative to the peptide HCVpc21 -1 (COCHO function at the N-terminal position).

5. Study of Sera with the Peptides HCVpc21 NS4 and an HCVpc21/NS4 Mixture

The peptide HCVpc21 is “spotted” in accordance with the protocol described in Example 4 at the concentration of 10⁴ M and the peptide NS4 is spotted at 10⁻³ M. For the mixture, the peptide NS4 is at a concentration of 0.5×10⁻³ M and the peptide HCVpc21 at a concentration of 0.5×10⁻⁴ M (10/1 ratio). Each peptide is spotted 16 times per slide. The results expressed correspond to the mean fluorescence of the 16 spots.

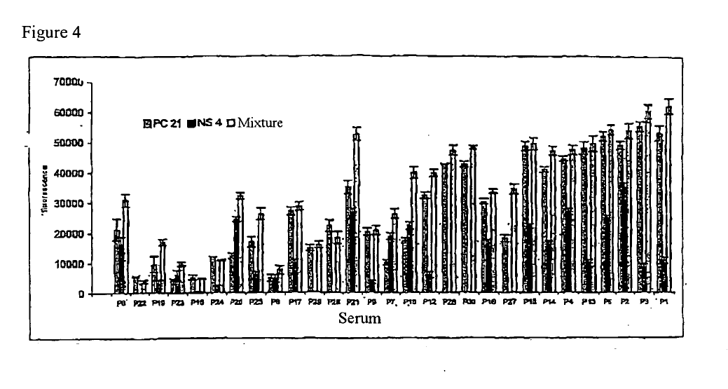

The results presented on FIG. 4 concern 30 referenced HCV sera which were positive according to the test from the Abbott company. These sera were numbered from P1 to P30.

The test according to the invention allowed the detection of all the positive sera. 10 negative control sera were tested, for which no fluorescence was detected (fluorescence corresponding to the background noise).

The test was thus found to be sensitive and 100% specific on the samples tested.

6. Test on Total Blood for the EBV Virus

On a semicarbazide glass slide, 16 spots of the EBV peptide (10⁻⁴ M) and 16 spots of the HCV peptide (10⁻⁴ M) were created.

A sample of fresh blood (patient negative for HCV and positive for EBV), of 100 μl taken from the finger tip was diluted in 50 μl of dilution buffer (PBS buffer, Tween 0.1%, semi-skimmed milk 2.5%). These 150 μl are then deposited on the slide and incubated for 2 hours at 37° C. under a cover slip.

The protocol for developing the slides is the same as described above.

Results: the HCV fluorescence is zero, the EBV fluorescence is 22991 (mean of 16 spots, standard deviation of 8.1% on these 16 spots).

7. Demonstration of the Sensitivity of Polypeptide Biochips

For a scanner power L35/PMT50, the background noise after contacting with the serum and the fluorescent antibody is on average 50, namely 0.1% of the maximum signal observed (statistical study on referenced 100 sera). This background noise is 16 times lower than that obtained on Carbobind® plates with the same peptides.

Moreover, for the negative sera, no fluorescence is observed at the level of the peptide spots, apart from that attributed to the background noise.

### EXAMPLE 6

### Utilisation of a Peptide Functionalised with a Ketone Group

A peptide derivative of HCVpc21 was solid phase synthesised from the N-terminal end on the basis of a Fmoc/tBu strategy, on an Fmoc-PAL-PEG-PS resin.

One part of the product (HCVpc2 1-Met) will be used as control:

H-MTNRRPQDVKFPGGGQIVGGVYLLPRRGPRLG-NH₂

One part of the product underwent a transamination to give the following derivative (HCVpc21-TA):

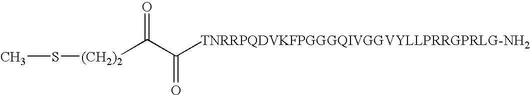

The reaction is effected according to the following protocol: One part of the above product (m=10.45 mg) is solubilised in 1165 μl of water; to this solution are added 1.165 ml of an aqueous solution at pH 5.5 containing: 2M sodium acetate, 0.2M acetic acid, 2.5 mM copper sulphate and 0.1M glyoxylic acid. After 5 hours of reaction, 1.165 ml of 7mM EDTA are added with stirring. The solution obtained is purified by preparative HPLC and the fractions corresponding to the product are lyophilised.

The 2 peptides HCVpc21-Met (non-transaminated) and pc21-TA (transaminated) and also the peptide HCVpc21-2 (described in Example 3) were attached to semicarbazide slides at the rate of 16 spots of each peptide per slide.

Each peptide was deposited at the concentration of 10⁻⁴ M.

The slides were incubated in the presence of 4 positive sera and 2 negative sera. The results presented in the following table show that

the transaminated peptide binds correctly to the semicarbazide slide,

the control peptide pc21-Met gives a weak response corresponding to a non-covalent (non-specific) adsorption.

TABLE II
Mean Fluorescence + (S.D. %)

| Peptides: | pc21-2 | pc21-TA | pc21-Met | Background noise |
|---|---|---|---|---|
| Serum 43 | 18158 | 12910 | 2810 | 50 |
| ELISA o.d. 1.7 | (5.9) | (6.3) | (10.2) | |
| Serum 45 | 968 | 1880 | 356 | 58 |
| ELISA o.d. 0.7 | (6.2) | (10.1) | (5.6) | |
| Serum 53 | 26963 | 23063 | 7741 | 56 |
| ELISA o.d. 2.7 | (7.1) | (6.8) | (7.8) | |
| Serum 41 | 6225 | 6169 | 960 | 61 |
| ELISA o.d. 1.4 | (5.5) | (6.5) | (5.0) | |
| Serum 48 | 58 | 58 | 59 | 57 |
| negative | | | | |
| Serum 55 | 56 | 54 | 55 | 54 |
| negative | | | | |

### EXAMPLE 7

### Attachment of a Glycosylated Protein to the Semicarbazide Slides

As a model for the attachment of a glycosylated protein to the semicarbazide slides, a fluorescent antibody is used, which enables it to be detected directly.

A rhodamine-conjugated anti-IgG-A-M antibody is oxidised to generate an aldehyde function using the procedure described by Wolfe and Hage (Anal. Bioch., 231, 123-130, 1995):

440 μi of antibody (1.5 mg/ml), diluted in a 0.02 M sodium acetate 0.15 M NaCl buffer of pH 5.5 were placed at 4° C. for 15 minutes. A 20 mM solution of sodium periodate in the same buffer is also placed at 4° C., protected from light. 275 μl of the sodium periodate solution are added to 275 μl of the antibody solution, protected from light, with stirring: solution A.

275 μl of the solution of 0.1 M sodium acetate 0.15 M NaCl buffer of pH 5.5 are added to 275 μl of the antibody solution, protected from light, with stirring: solution B.

The two solutions are placed at 4° C. for 20 minutes. Then 137 μl of ethylene glycol (0.25 ml per millilitre of sample) are added to each of them with stirring.

These two solutions are then transferred into a dialysis tube (Nanosep, Microconcentrators, Gelman Sciences, Filtron Brand).

The excess of reagent is eliminated by a series of 4 centrifugations effected without drying the membrane (3,000 revolutions per minute, 60 minutes, then 10,000 revolutions per minute, 20 minutes), rediluting each time with the pH 5.5 acetate buffer solution (addition of 200 μl of buffer).

These two solutions were sampled using a 1 μl minicaps (Hirschmann Laborgerate) and spotted 8 times onto a glass slide ftinctionalised with semicarbazide groups as previously described.

The slide is then placed to incubate for 6 hours at 37° C. under a moist atmosphere. It is then washed by immersion in a 0.1 M solution of Tris acetate with 0.1% Tween 20 for 60 minutes with ultrasonication. The slide is then rinsed with water and dried with ethanol. The fluorescence is made visible in the slide scanner.

The fluorescence values obtained for the oxidised antibody (solution A) and the non- oxidised antibody (solution B) are presented in the following table:

| | Fluorescence | Standard Deviation (%) |
|---|---|---|
| Oxidised antibody | 26,270 | 9.8 |
| Non-oxidised antibody | 6,068 | 14.5 |
| Background noise | 141 | |

### EXAMPLE 8

### Epitopic Screening

Synthesis:

A chemical library of 24 decapeptides was synthesised in parallel so as to cover 3 loops of the protein NS3 exposed to the solvent. The synthesis was carried out in solid phase from the C-terminal end towards the N-terminal end on the basis of the standard Fmoc/tert-butyl strategy and in situ PyBOP/DIEA activation on the resin “Gly-4,7,10-trioxa-1,13-diamino -tridecanyl-methyl-2′,3′-O-isopryldene-D-tartryl-Val-PEGA”, prepared as described for the peptide HCVpc21-2.

The synthesis of the 24 peptides was carried out on a 96-channel multiple automatic synthesiser of the ACT 496 MOS type from Advanced Chemtech. The syntheses were done on a 0.02 mole scale and 15 eq. of activated acid were used for each coupling stage (simple couplings). The peptidyl resins were acetylated with the mixture Ac₂O/DIEA/NMP (3/0.3/86.7) after each coupling. At the end of the synthesis, the resins were transferred in syringes equipped with ffitted plates (ABIMED brand) and deprotected at ambient temperature with 1 ml of the mixture TFA/H₂O/ethanedithiol (95/2.5/2.5 by vol.) for 2 hours. The resins were then washed with CH₂Cl₂, methanol and finally ethyl ether and dried. The resins were conditioned in a 10% solution of acetic acid in water, then treated with 6 eq. of NaIO₄ (25.7 mg) dissolved in 500 μl of water for 2 minutes. The oxidative cleavage was stopped by addition of 24 equivalents of ethanolamine (29 μl). After two washings with 1 ml of water, the filtrates were mixed and desalted by RP HPLC (C3-Zorbax column, 0% B to 100% B, flow rate 5 ml.min^{31 1}, detection at 230 nm). After lyophilisation, the peptides were analysed by analytical RP-HPLC and by MALDI-TOF mass spectrometry. The results obtained are brought together in the following table.

The general formula of the peptides is shown in the figure below.

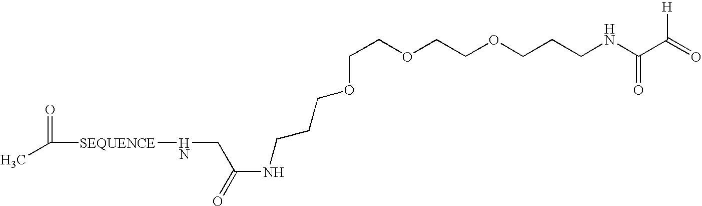

| | | [3] | [4] | | [5] |
|---|---|---|---|---|---|
| [1] | [2] | Rdt | Pureté | [M + H]⁺ | m/z observé |
| Peptide | Sequences | % | HPLC | calc. | Pic Principal |
| 1 | YGKAIPLEAI | 30 | 89% | 1431.67 | 1453.64 (+Na) |
| 2 | GKAIPLEAIK | 32 | 90% | 1396.67 | 1418.75 (+Na) |
| 3 | KAIPLEAIKG | 30 | 91% | 1396.67 | 1418.77 (+Na) |
| 4 | AIPLEAIKGG | 30 | 80% | 1325.54 | 1347.70 (+Na) |
| 5 | IPLEAIKGGR | 30 | 76% | 1410.65 | 1410.99 |
| 6 | PLEAIKGGRH | 35 | 31% | 1434.63 | 1435.98 (+H) |
| 7 | LEAIKGGRHL | 31 | 76% | 1450.68 | 1451.02 |
| 8 | EAIKGGRHLL | 34 | 75% | 1450.68 | 1451.98 (+H) |
| 9 | ELAAKLSGLG | 32 | 72% | 1315.50 | 1337.86 (+Na) |
| 10 | LAAKLSGLGI | 27 | 75% | 1299.55 | 1321.91 (+Na) |
| 11 | AAKLSGLGIN | 30 | 72% | 1300.49 | 1322.87 (+Na) |
| 12 | AKLSGLGINA | 29 | 72% | 1300.49 | 1322.86 (+Na) |
| 13 | KLSGLGINAV | 23 | 72% | 1328.55 | 1350.87 (+Na) |
| 14 | LSGLGINAVA | 29 | 65% | 1271.45 | 1293.S8 (+Na) |
| 15 | SGLGINAVAY | 60 | 86% | 1321.47 | 1343.83 (+Na) |
| 16 | GLGINAVAYY | 6 | 15% | 1397.57 | 1419.90 (+Na) |
| 17 | GLDVSVIPTS | 29 | 36% | 1344.50 | 1366.84 (+Na) |
| 18 | LDVSVIPTSG | 28 | 78% | 1344.5 | 1366.85 (+Na) |
| 19 | DVSVIPTSGD | 26 | 63% | 1346.43 | 1368.80 (+Na) |
| 20 | VSVIPTSGDV | 28 | 60% | 1330.47 | 1352.77 (+Na) |
| 21 | SVIPTSGDVV | 30 | 64% | 1330.47 | 1352.67 (+Na) |
| 22 | VIPTSGDVVV | 26 | 86% | 1342.53 | 1364.70 (+Na) |
| 23 | IPTSGDVVVV | 21 | 40% | 1342.53 | 1364.68 (+Na) |
| 24 | PTSGDYVVVA | 17 | 46% | 1300.45 | 1322.65 (+Na) |

Key:
[1] peptide;
[2] sequences;
[3] yield. %;
[4] HPLC purity;
[5] m/z observed, main peak.

Test on Biochip:

These peptive derivatives of the NS3 protein of HCV were printed onto a semicarbazide glass slide and tested with 4 referenced NS3+sera (Recombinant Immunoblot Assay, DECISCAN HCV PLUS) and one referenced HCV serum with the same test as indicated in Example 4 (Protocol for utilisation of the semicarbazide slides).

This experiment makes it possible to identify, in the collection of 24 peptides, those which are of most interest for the detection of antibodies directed against NS3.

The serodetection results collected in the table below show that the peptides 14 and 15 are of intrest for the detection of anti-NS3 antibodies, and that in general the peptide biochips constitute a valuable tool for the screening of epitopes.

| | [1] Sérums | | | | |
|---|---|---|---|---|---|
| | [2] Sérums NS3 positifs | | | | [3] |
| Peptides | Sérum 1 | Sérum 2 | Sérum 3 | Sérum 4 | Sérum HCV- |
| 1 | 3735 | 3143 | 2608 | 6357 | 917 |
| 2 | 3590 | 8023 | 3495 | 7405 | 933 |
| 3 | 2803 | 3161 | 1694 | 5065 | 924 |
| 4 | 2899 | 2988 | 2497 | 5598 | 1011 |
| 5 | 2260 | 2339 | 1549 | 4739 | 945 |
| 6 | 2789 | 3136 | 2821 | 5932 | 957 |
| 7 | 1845 | 1307 | 1084 | 3077 | 574 |
| 8 | 2343 | 1220 | 1159 | 2587 | 525 |
| 9 | 1582 | 1142 | 1671 | 2418 | 508 |
| 10 | 3166 | 2418 | 1684 | 9152 | 551 |
| 11 | 2156 | 1557 | 1458 | 5283 | 628 |
| 12 | 5209 | 1619 | 1780 | 6123 | 605 |
| 13 | 20122 | 5668 | 19302 | 6400 | 990 |
| 14 | 17128 | 8922 | 20216 | 6198 | 1012 |
| 15 | 17944 | 10603 | 4786 | 8262 | 791 |
| 16 | 4201 | 5577 | 1861 | 5602 | 931 |
| 17 | 7976 | 6423 | 2619 | 7338 | 754 |
| 18 | 29857 | 4113 | 4824 | 6209 | 667 |
| 19 | 9088 | 4111 | 2337 | 4939 | 567 |
| 20 | 7909 | 4711 | 2873 | 11266 | 718 |
| 21 | 3553 | 8321 | 2244 | 6853 | 913 |
| 22 | 1882 | 1466 | 1385 | 2963 | 648 |
| 23 | 2983 | 3394 | 1818 | 4505 | 904 |
| 24 | 1835 | 1436 | 1387 | 3532 | 573 |
| [4] BF | 1425 | 1708 | 1512 | 1622 | 701 |
| [5] ecart-type (ec) | 260 | 619 | 954 | 384 | 190 |
| [6] Seuil de | 2205 | 3565 | 4374 | 2774 | 1271 |
| positivité | | | | | |
| (BF + 3ec) | | | | | |

Key:
[1] sera;
[2] NS3 positive sera;
[3] HCV serum;
[4] background noise;
[5] standard deviation (S.D.);
[6] positivity threshold (background noise + 3 S.D.).

### EXAMPLE 9

### Study of Ligand-Receptor Relationships

The Ligand is a Biotinylated Peptide and the Receptor is Streptavidin or an Anti-Biotin Antibody.

Synthesis of the Peptide

H-Lys(biotin-G₂)AYLAG-NH(CH₂)₃O(CH₂)₂O(CH₂)₂O(CH₂)₃NHCOCHO

The syntesis was carried out on Novasyn TGR resin (Novabiochem, lot A 23633, loading 0.2 mmole/g) on a 0.1 mmole scale.

This resin was treated as described for the peptide HCVpc21-2, the PEGA resin being replaced by the resin Novasyn TGR. After the coupling of the Fmoc-Gly-OH, the syntesis was continued on a Perseptive Biosystems Pionneer synthesiser, using the Fmoc/tert-butyl strategy. The following acids were coupled successively (10 eq., simple coupling, TBTU/HOBt/DIEA activation): Fmoc-L-Ala-OH, Fmoc-L-Leu-OH, Fmoc-L-Val-OH, Fmoc-L-Tyr (OtBu)-OH, Fmoc-L-Ala-OH, Boc-L-Lys(Fmoc)-OH, Fmoc-Gly-OH twice, biotin.

The deprotection and the cleavage of the peptide from the resin was effected with 10 ml of the mixture TFA 95/H₂O 2.5/dimethyl sulphide 2.5 (1 hr at ambient temperature). The peptide was precipitated in ethyl ether/pentane: 1/1 (by vol.), centrifuged, taken up again in 30% AcOH in water and lyophilised (69 mg). The peptide was purified on a C18 Nucleosil column (A: H₂O 0 0.05% TFA, B: CH₃CN/H₂O: 4/1 by vol., 0.05% TFA 0-25% 20 mins, 25-25% 5 mins, 25-35% 40 mins). 35.72 mg of pure product are obtained.

The oxidation is performed with 6 eq. of NaIO₄ and 8 eq. of methionine at a concentration of 0.5 mM in 100 mM phosphate pH 6.6/MeOH: 1/1 by vol. for 15 mins. The reaction is stopped with 12 eq. of ethanolamine, diluted with water and purified on a C18 Nucleosil column (0-23% 20 mins, 23-23% 5 mins, 23-35% 35 mins) to give the expected product.

Test on Biochips

The pep-biotin peptide is “spotted” according to the protocol described in Example 4 paragraph 1 at concentrations of 10⁻³M, 10⁻⁴M, 10⁻⁵M and 10⁻⁶M. Each concentration is spotted 6 times per slide.

The slides are then incubated in the presence of 150 μl of ligand (solution of Rhodamine Conjugated affinity Purified anti-Biotin [goat] (Rockland, Gilbertsville, Pa., USA) or solution of Streptavidin Tetramethyl Rhodamine conjugate (Molecular Probes, Oregon, USA)) diluted in an 0.01 M PBS buffer, pH 7.2 (ligand concentrations: 10⁻¹ mg/ml, 10⁻² mg/ml, 10⁻³ mg/ml, 10⁻⁴ mg/ml) under a glass cover slip (24×60 mm). The slides are incubated for 2 hours at 37° C. under a moist atmosphere. A series of 4 successive washings is carried out with PBS solution to which 0.05% Tween 20 had been added. The slides are then rinsed with a deionised water wash-bottle then with 95° ethanol for 1 minute, then dried for 15 minutes in the ambient atmosphere.

The fluorescence is then detected using a slide scanner (L35/PMT50, Affymetrix 418 Array Scanner, MWG). The results expressed in the following table correspond to the mean fluorescence and to the standard deviation of the 6 spots of each concentration obtained after incubation of the rhodamine-conjugated streptavidin and the rhodamine- conjugated anti-biotin antibody respectively.

[1] [Streptavidine rhodaminée]

| | 10⁻¹ mg/ml [3] | | 10⁻² mg/ml | | 10⁻³ mg/ml | | 10⁻⁴ mg/ml | |
|---|---|---|---|---|---|---|---|---|
| [pep-Biotine] | Moyenne | Ecart type | Moyenne | Ecart type | Moyenne | Ecart type | Moyenne | Ecart type |
| 10⁻³ mg/ml | 34660 | 2540 | nd | nd | 29836 | 1515 | 13816 | 4039 |
| 10⁻⁴ mg/ml | 31781 | 3056 | nd | nd | 24666 | 2886 | 7931 | 1435 |
| 10⁻⁵ mg/ml | 26524 | 2095 | nd | nd | 20902 | 2554 | 7673 | 818 |
| 10⁻⁶ mg/ml | 18500 | 1916 | nd | nd | 10737 | 887 | 2124 | 733 |
| Bruit de fond | 10062 | 515 | nd | nd | 580 | 337 | 174 | 36 |

[2] [Ac anti-biotine rhodaminée]

| | 10⁻¹ mg/ml | | 10⁻² mg/ml | | 10⁻³ mg/ml | | 10⁻⁴ mg/ml | |
|---|---|---|---|---|---|---|---|---|
| [pep-Biotine] | Moyenne | Ecart type | Moyenne | Ecart type | Moyenne | Ecart type | Moyenne | Ecart type |
| 10⁻³ mg/ml | 15350 | 1172 | 13113 | 637 | 6828 | 883 | 2271 | 348 |
| 10⁻⁴ mg/ml | 14721 | 1443 | 9121 | 1974 | 4981 | 1065 | 1535 | 511 |
| 10⁻⁵ mg/ml | 10932 | 1475 | 6849 | 933 | 3377 | 292 | 1249 | 67 |
| 10⁻⁶ mg/ml | 6606 | 125 | 2947 | 825 | 791 | 87 | 385 | 39 |
| [4] Bruit de fond | 1424 | 109 | 243 | 13 | 167 | 19 | 170 | 12 |

Key:
[1] rhodamine-conjugated streptavidin;
[2] rhodamine-conjugated antibiotin antibody;
[3] mean, standard deviation (each pair of columns);
[4] background noise.

Study of competition for biotin showing the specificity of the ligand-receptor interaction:

The peptide pep-biotin is “spotted” according to the protocol described in Example 4 paragraph 1 at the concentrations of 10⁻³M, 10⁻⁴M, 10⁻⁵M and 10⁻⁶M. Each concentration is spotted 6 times per slide.

The slides are then incubated in the presence of 150 μl of a 10⁻³ mg/ml solution of rhodamine-conjugated streptavidin (solution of streptavidin Tetramethyl Rhodamine conjugate (Molecular Probes, Oregon, USA)) diluted in an 0.01 M PBS buffer, pH 7.2 in 15 the presence of different concentrations of biotin (10⁻³ M, 10⁻⁴ M, 10⁻⁵ M, 10⁻⁷ M and 0 M under a glass cover slip (24×60 mm). The slides are incubated for 2 hours at 37° C. under a moist atmosphere. A series of 4 successive washings is carried out with PBS solution to which 0.05% Tween 20 had been added. The slides are then rinsed, dried and analysed as stated above. The results expressed in the following table correspond to the mean fluorescence and to the standard deviation of the 6 spots of each concentration.

[Biotin] in competition

| [pep- | 0 M | | 10⁻⁷ M | | 10⁻⁶ M | | 10⁻⁴ M | | 10⁻³ M | |
|---|---|---|---|---|---|---|---|---|---|---|
| Biotine] | M | ET | M | ET | M | ET | M | ET | M | ET |
| 10⁻³ mg/ml | 29836 | 1515 | 17132 | 7141 | 16683 | 1615 | 2237 | 394 | 376 | 78 |
| 10⁻⁴ mg/ml | 24666 | 2886 | 12178 | 4067 | 9414 | 686 | 961 | 65 | 207 | 25 |
| 10⁻⁵ mg/ml | 20902 | 2554 | 6129 | 1701 | 3588 | 840 | 322 | 42 | 107 | 10 |
| 10⁻⁶ mg/ml | 10737 | 887 | 2611 | 441 | 2350 | 496 | 189 | 8 | 78 | 13 |
| [1] Bruit de | 580 | 337 | 116 | 15 | 146 | 8 | 95 | 7 | 75 | 6 |
| fond | | | | | | | | | | |

M: moyenne de fluorescence
ET: écart-type sur la fluorescence
Key:
[1] background noise;
M: mean fluorescence,
ET: standard deviation on fluorescence.

1. Device for presentation of peptides, able to be used as a “chip” for a miniaturised detection of structurally or functionally complementary molecules, and consisting of a flat support onto which the said polypeptides are covalently bonded, characterised in that the bonding between the polypeptides and the support results from the formation of a semicarbazone bond.

2. Device according to claim 1, characterised in that the semicarbazone bond results from the reaction between
polypeptides bearing an aldehyde or ketone function
and a support functionalised with semicarbazide groups.

3. Device according to claim 1, characterised in that the support is constituted of a solid material, organic or inorganic, of the type: glass, silicon or derivatives thereof, or synthetic polymers, and presenting a flat surface.

4. Device according to claim 2, characterised in that the flat support is densely, homogeneously and reproducibly functionalised with semicarbazide groups.

5. Device according to claim 2, characterised in that the flat support is densely, homogeneously and reproducibly functionalised with semicarbazide groups and that the quality of the said functionalisation of the support (density and homogeneity) is checked by its capacity to bind a synthetic fluorescent peptide probe, derivatised with an α-oxo aldehyde function.

6. Device according to claim 2, characterised in that the aldehyde or ketone functions carried by the polypeptides are α-oxo aldehyde or α-oxo ketone groups, and are located on the C-terminal side or the N-terminal side, or on a side-chain.

7. Device according to claim 2, characterised in that the aldehyde or ketone functions carried by the polypeptides are α-oxo aldehyde or α-oxo ketone groups, and are situated on a spacer arm located whether on the C-terminal side or on the N-terminal side, or on a side-chain.

8. Process for preparation of a device according to claim 2, characterised in that it comprises the following stages:
1—introduction of an aldehyde or ketone function, by synthesis or by modification of a natural function, at one of the ends N or C or on a side-chain of a synthetic or natural polypeptide;
2—functionalisation of a solid support with semicarbazide groups;
3—deposition in the form of spots of samples of polypeptides obtained by stage 1 onto the support functionalised in stage 2, under pH and humidity conditions ensuring the reaction between the aldehyde or ketone function and the semicarbazide function to create the semicarbazone bond.

9. Process according to claim 8, characterised in that stage 1 is effected in the course of the synthesis of a polypeptide using an automatic synthesiser.

10. Process according to claim 8, characterised in that stage 1 further includes the introduction of a spacer arm between the last amino acid of the polypeptide sequence and the aldehyde or ketone function.

11. Process according to claim 8, characterised in that stage 1 is effected by oxidation of a polysaccharide of a natural glycoprotein or of a fragment thereof

12. Process according to claim 8, characterised in that stage 1 is effected by transamination of an N-terminal amino acid of a natural protein, whether or not glycosylated, or of a fragment thereof.

13. Process according to claim 8, characterised in that stage 2 comprises
a reaction of silanisation of the support, introducing an amine function;
the transformation of the amine function into an isocyanate function;
the reaction of the isocyanate function with a hydrazine derivative to form the semicarbazide group.

14. Process according to claim 8, characterised in that stage 2 is effected in a single reaction of a silane bearing a semicarbazide group.

15. Process according to claim 8, characterised in that stage 2 is effected in a single reaction of a silane bearing a semicarbazide group which is protected with Fmoc.

16. Process according to claim 8, characterised in that stage 3 comprises
the preparation of 10⁻³ or 10⁻⁴ M solutions of the polypeptides from stage 1 in an 0.1 M acetate buffer at pH 5.5,
the distribution of said solutions in a receptacle appropriate for their sampling, of the microtitration well slide type,
the sampling of said solutions using a “spotter”,
and the deposition of said solutions onto the semicarbazide support;
the incubation of the slides for one night at 37° C. under a moist atmosphere
and the washing of the slides and the saturation of the non-specific reactive sites.

17. Utilisation of devices according to claim 1 as polypeptide chips as a diagnostic tool.

18. Utilisation of devices according to claim 1 as polypeptide chips as a diagnostic tool, characterised in that it comprises the detection of responses of the antigen-antibody type by the utilisation of labelled, fluorescent, radioactive or chemically labelled reagents, as in non-miniaturised diagnostic tests.

19. Utilisation of devices according to claim 1 as polypeptide chips for the screening of molecules.

20. Utilisation of devices according to claim 1 as polypeptide chips for the analysis of the relationships between molecules, of the ligand-receptor type.
